(19) United States
(12) Patent Application Publication
(10) Pub. No.: US 2016/0189520 A1
(43) Pub. Date: Jun. 30, 2016

(54) Electronic Device Covers Having Gas Sensors

(71) Applicant: SPEC Sensors, LLC, Newark, CA (US)

(72) Inventors: Marc Papageorge, Pleasanton, CA (US); Bennett Meulendyk, Dublin, CA (US); Joseph R. Stetter, Hayward, CA (US); Vinay Patel, Fremont, CA (US); David Peaslee, Newark, CA (US); Gavin O'Toole, Newark, CA (US)

(73) Assignee: SPEC Sensors, LLC, Newark, CA (US)

(21) Appl. No.: 14/985,674

(22) Filed: Dec. 31, 2015

Related U.S. Application Data
(60) Provisional application No. 62/098,969, filed on Dec. 31, 2014.

(51) Int. Cl. G08B 21/12; G01N 33/00

(52) U.S. Cl. CPC G08B 21/12; G01N 33/0063; G01N 33/0036

(57) ABSTRACT

An electronic device cover system that includes an electronic device cover engageable with an electronic device, a gas sensor coupled to the electronic device cover, and a control circuit communicatively coupled to the gas sensor and communicatively engageable with an electronic device. When the gas sensor detects a presence of a target gas, the control circuit receives a signal output by the gas sensor and outputs a signal receivable by an electronic device.

### CROSS-REFERENCE TO RELATED APPLICATIONS

This application claims the benefit of U.S. Provisional Application Ser. No. 62/098,969, filed Dec. 31, 2014.

### TECHNICAL FIELD

The present specification generally relates to an electronic device cover interfaced with one or more gas sensors, for example, microelectronic gas sensors, printed gas sensors, or the like.

### BACKGROUND

Sensors including electrochemical cells are used for detection of certain gases, for example, toxic gases and gases in a person's breath. Accordingly, electronic device covers are desired that include sensors used for detection of gases to communicatively couple these sensors with electronic devices.

### SUMMARY

In one embodiment, an electronic device cover system includes an electronic device cover engageable with an electronic device, a gas sensor coupled to the electronic device cover, and a control circuit communicatively coupled to the gas sensor and communicatively engageable with an electronic device. When the gas sensor detects a presence of a target gas, the control circuit receives a signal output by the gas sensor and outputs a signal receivable by an electronic device.

In another embodiment, an electronic device cover system includes an electronic device cover, a gas sensor and a control circuit. The electronic device cover is engageable with an electronic device. The electronic device cover includes a sensor housing portion engageable with a sensor cover portion. The gas sensor is coupled to the sensor housing portion of the electronic device cover. The sensor cover portion of the electronic device cover comprises a gas access hole extending through the sensor cover portion such that when the sensor cover portion is engaged with the sensor housing portion, the gas access hole is fluidly coupled to the gas sensor. Further, the gas sensor is a printed gas sensor including a substrate layer having one or more gas access regions fluidly coupled to the gas access hole of the sensor cover portion, one or more printed runners coupled to the substrate layer, where the one or more printed runners are electrically conductive, an encapsulation layer coupled to the substrate layer and defining an electrolyte cavity positioned within the encapsulation layer, one or more electrodes positioned in electrical communication with the one or more printed runners such that the one or more printed runners can transport an electronic signal produced by an electrochemical reaction at the one or more electrodes, and an electrolyte housed within the electrolyte cavity. Further, the control circuit is communicatively coupled to the one or more printed runners of the gas sensor and communicatively engageable with an electronic device such that when the gas sensor detects a presence of a target gas, the control circuit receives a signal output by the gas sensor and outputs a signal receivable by an electronic device.

In yet another embodiment, a electronic device cover system for generating sensor feedback including an electronic device cover engageable with an electronic device, a gas sensor coupled to the electronic device cover and communicatively coupled to one or more processors, one or more memory modules communicatively coupled to the one or more processors, and machine readable instructions stored in the one or more memory modules that, when executed by the one or more processors, causes the one or more processors to receive sensor information from the gas sensor, generate feedback regarding a presence of a target gas based on sensor information received from the gas sensor, and output feedback regarding the presence of the target gas using the electronic device.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an example electronic device cover system including an electronic device cover and an electronic device according to one or more embodiments shown or described herein;

FIG. 2 schematically depicts communicatively coupled electrical components of the electronic device cover system according to one or more embodiments shown and described herein;

FIG. 3 schematically depicts a sensor housing portion of the electronic device cover of FIG. 1 and a plurality of gas sensors coupled thereto according to one or more embodiments shown or described herein;

FIG. 4 schematically depicts a device engaging portion of the electronic device cover of FIG. 1 and a plurality of gas sensors coupled thereto according to one or more embodiments shown or described herein;

FIG. 5 schematically depicts an exploded side sectional view of the electronic device and electronic device cover of FIG. 1 according to one or more embodiments shown and described herein; and

FIG. 6 schematically depicts a sectional view of an example gas sensor of the electronic device cover system of FIGS. 1-5 according to one or more embodiments shown or described herein.

### DETAILED DESCRIPTION

The present disclosure relates to an electronic device cover system that includes covers and cases for electronic devices interfaced with one or more gas sensors, for example, microelectronic gas sensors, printed gas sensors, or any known gas sensor in the art. The electronic device cover system of the present disclosure may communicatively couple various gas sensors with an electronic device to provide the electronic device with the functionality of the gas sensors. Further, the gas sensors may be coupled to, for example, integrated into an electronic device cover that may also provide physical protection to both the electronic device and the gas sensors and control circuit coupled to the electronic device cover.

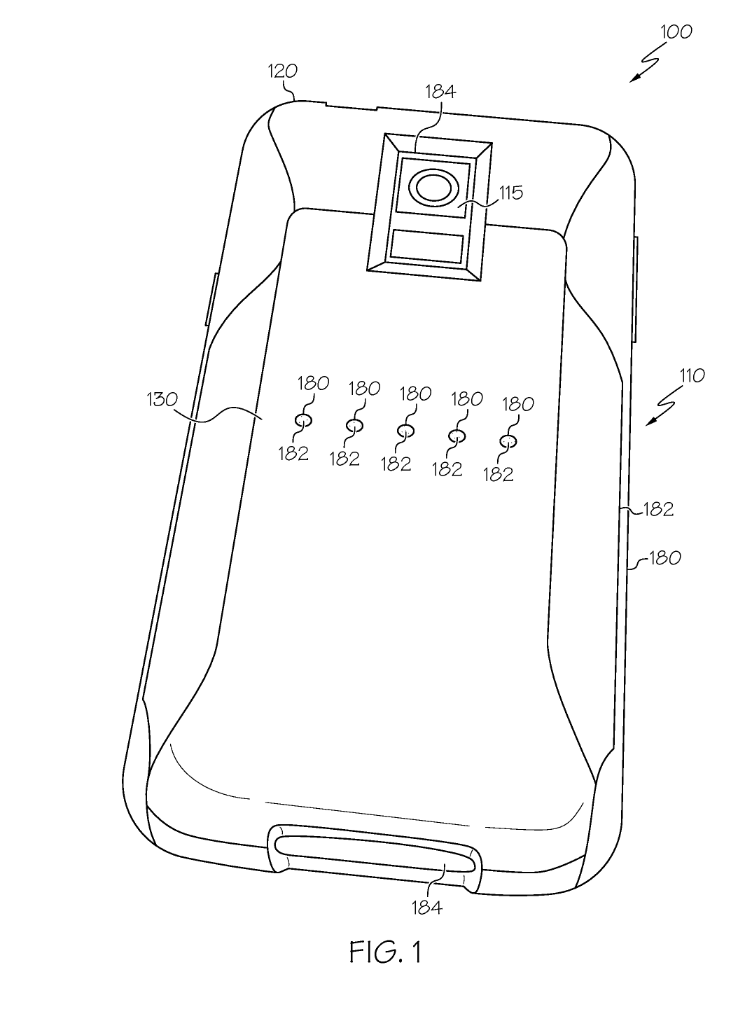

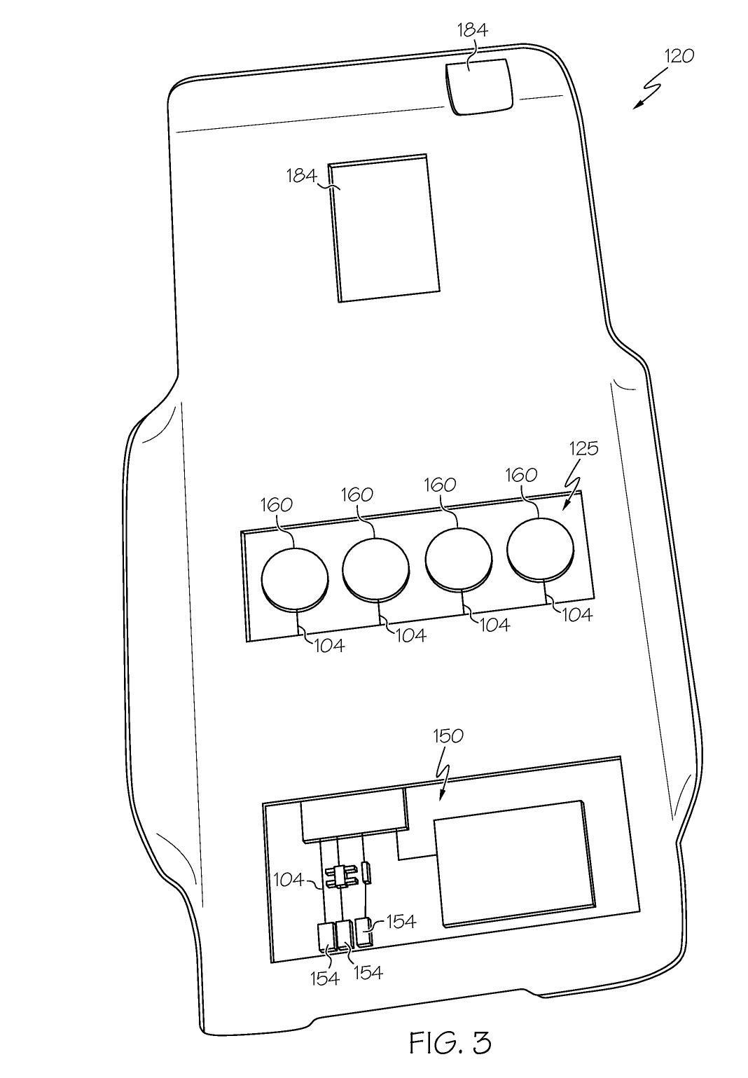

Referring to FIG. 1, an electronic device cover system 100 comprises an electronic device cover 110, one or more gas sensors 160 (FIG. 3) coupled to the electronic device cover 110, and a control circuit 150 (FIG. 3) electrically and communicatively coupled to the one or more gas sensors 160 (FIG. 3) to communicatively couple the one or more gas sensors 160 to an electronic device 115. In some embodiments, the control circuit 150 may be coupled to the electronic device cover 110 to facilitate communication between the gas sensor 160 and the electronic device 115. In other embodiments, the control circuit 150 may be part of the electronic device 115, allowing the electronic device to communicate directly with the one or more gas sensors 160. The electronic device cover 110 may be a cover and/or a case configured to be coupled to any electronic device 115, for example cell phones, smartphones, tablets, laptops, GPS devices, watches, handset devices, electronic badges (e.g., tracking badges) and other mobile and/or wearable electronic technology, for example, electronic products of known brand providers such as iPhone™, Galaxy™, HTC™, LG™, Motorola™, and the like.

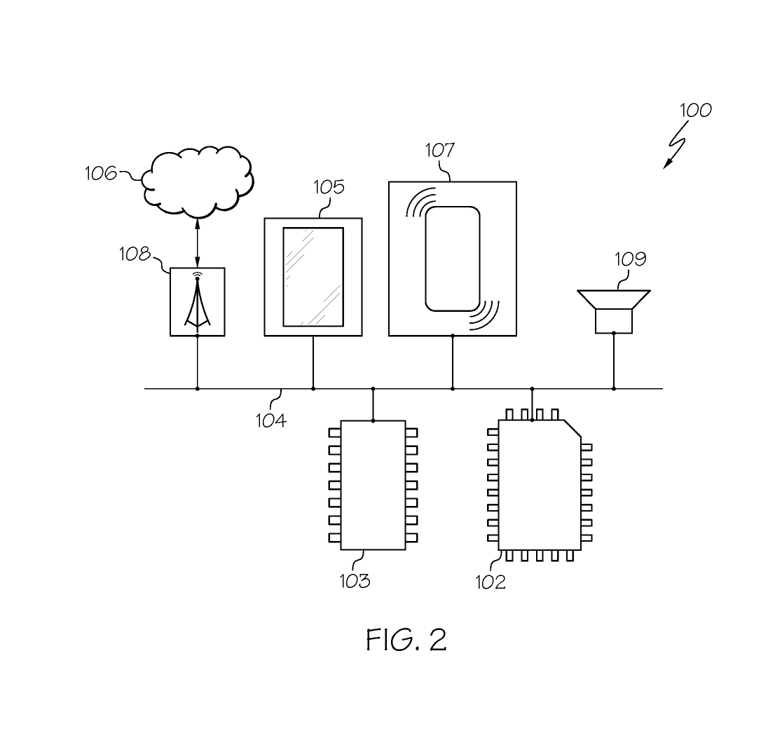

Referring now to FIG. 2, electrical components of the electronic device cover system 100 are schematically depicted. As depicted in FIG. 2, the electronic device cover system 100 comprises one or more processors 102, for example, the control circuit 150 may comprise one or more processors 102. Each of the one or more processors 102 may be any device capable of executing machine readable instructions, for example, a controller, an integrated circuit, a microchip, a computer, or any other computing device. The one or more processors 102 are coupled to a communication path 104 that provides signal interconnectivity between various electrical components of the electronic device cover system 100. Accordingly, the communication path 104 may communicatively couple any number of processors 102 with one another, and allow the electronic components coupled to the communication path 104 to operate in a distributed computing environment. Specifically, each of the electronic components may operate as a node that may send and/or receive data. As used herein, the term “communicatively coupled” means that coupled electronic components are capable of exchanging data signals with one another such as, for example, electrical signals via conductive medium, electromagnetic signals via air, optical signals via optical waveguides, and the like.

The communication path 104 may be formed from any medium that is capable of transmitting a signal such as, for example, conductive wires, conductive traces, optical waveguides, or the like. In some embodiments, the communication path 104 may facilitate the transmission of wireless signals, such as wireless fidelity (Wi-Fi), Bluetooth, Bluetooth low energy, and the like. Moreover, the communication path 104 may be formed from a combination of mediums capable of transmitting signals. For example, the communication path 104 may comprise a combination of conductive traces, conductive wires, connectors, and buses that cooperate to permit the transmission of electrical data signals to components such as processors, memories, sensors (e.g., the gas sensors 160), input devices, output devices, and communication devices. Further, the communication path 104 may provide a communications pathway to transmit sensor information output by the gas sensor 160 and/or the control circuit 150 to the electronic device 115.

The electronic device cover system 100 may further comprise one or more memory modules 103 coupled to the communication path 104, for example, the control circuit 150 and/or the electronic device 115 may comprise one or more memory modules 103. In some embodiments, the one or more memory modules 103 may comprise cloud based memory. Further, the one or more memory modules 103 may comprise RAM, ROM, flash memories, hard drives, or any device capable of storing machine readable instructions such that the machine readable instructions can be accessed by the one or more processors 102. The machine readable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable instructions and stored on the one or more memory modules 103. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

Still referring to FIG. 2, in some embodiments, the electronic device cover system 100 including the control circuit 150, the one or more gas sensors 160, and the electronic device 115 may be communicatively coupled by a network 106. In one embodiment, the network 106 is a personal area network that utilizes Bluetooth technology to communicatively couple the control circuit 150, the one or gas sensors 160, and the electronic device 115. In other embodiments, the network 106 may include one or more computer networks (e.g., a personal area network, a local area network, or a wide area network), cellular networks, and/or satellite networks, and combinations thereof. Accordingly, the electronic device cover system 100 can be communicatively coupled to the network 106 via wires, via a wide area network, via a local area network, via a personal area network, via a cellular network, via a satellite network, etc. Suitable local area networks may include wired Ethernet and/or wireless technologies such as, for example, Wi-Fi. Suitable personal area networks may include wireless technologies such as, for example, IrDA, Bluetooth, Wireless USB, Z-Wave, ZigBee, and/or other near field communication protocols. Suitable personal area networks may similarly include wired computer buses such as, for example, USB and FireWire. Suitable cellular networks include, but are not limited to, technologies such as LTE, WiMAX, UMTS, CDMA, and GSM.

Still referring to FIG. 2, the electronic device cover system 100 comprises network interface hardware 108 for communicatively coupling the control circuit 150, the one or more gas sensors 160, and the electronic device 115. The network interface hardware 108 may be communicatively coupled to the communication path 104 and can be any device capable of transmitting and/or receiving data via a network. Accordingly, the network interface hardware 108 can include a communication transceiver for sending and/or receiving any wired or wireless communication. For example, the network interface hardware 108 may include an antenna, a modem, LAN port, Wi-Fi card, WiMax card, mobile communications hardware, near-field communication hardware, satellite communication hardware and/or any wired or wireless hardware for communicating with other networks and/or devices. In one embodiment, the network interface hardware 108 includes hardware configured to operate in accordance with the Bluetooth wireless communication protocol and may include a Bluetooth send/receive module for sending and receiving Bluetooth communications. Further, in some embodiments, the control circuit 150 is configured with wired and/or wireless communication functionality for communicating with the one or more gas sensors 160, and the electronic device 115. For example, the control circuit 150 may include a communication transceiver for sending and/or receiving any wired or wireless communication. Moreover, in operation, the one or more processors 102, the one or more memory modules 103, the communications path 104, the network 106, and the network interface hardware 108 may perform one or more handshake protocols when communicatively coupling the electronic device 115 and the one or more gas sensors 160.

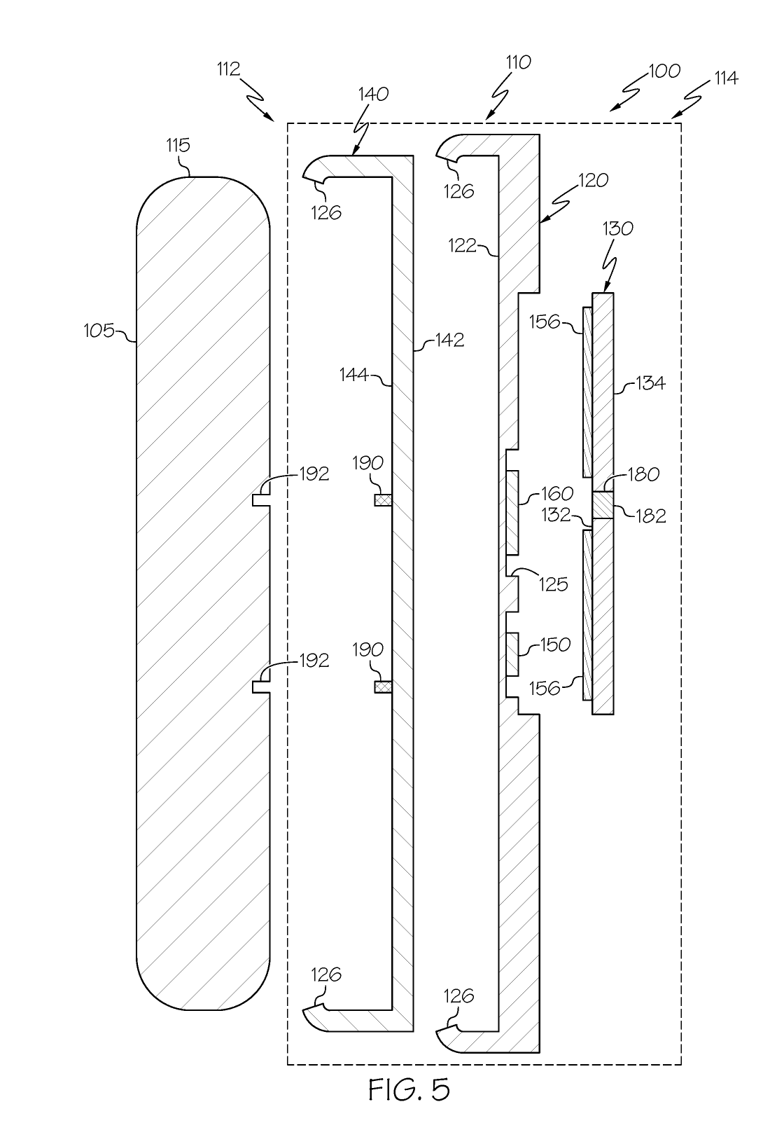

Referring still to FIG. 2, the electronic device cover system 100 may further comprise a display 105 for providing visual output, for example, sensor information and other visual output. The display 105 may be positioned on the electronic device 115 (FIG. 5) and may be positioned on one or multiple sides of the electronic device 115, for example, positioned on both a “front” side and a “back” side of the electronic device 115. Further, the display 105 is coupled to the communication path 104. Accordingly, the communication path 104 communicatively couples the display 105 to other electrical components of the electronic device cover system 100. The display 105 may include any medium capable of transmitting an optical output such as, for example, a cathode ray tube, light emitting diodes, a liquid crystal display, a plasma display, or the like. Moreover, the display 105 may be a touchscreen that, in addition to providing optical information, detects the presence and location of a tactile input upon a surface of or adjacent to the display.

Still referring to FIG. 2, the electronic device cover system 100 may further comprise one or more auditory devices 109, for example, speakers, coupled to the communication path 104 such that the communication path 104 communicatively couples the one or more auditory devices 109 to other electrical components of the electronic device cover system 100. For example, the one or more auditory devices 109 may be embedded within the electronic device 115. The one or more auditory devices 109 transform data signals from the electronic device cover system 100 into audible mechanical vibrations. In some embodiments, the one or more auditory devices 109 may be configured to provide audible information regarding the measurements of the gas sensor 160, such as, for example, an alarm, a vocal message, or the like.

Still referring to FIG. 2, the electronic device cover system 100 may further comprise one or more tactile feedback devices 107 communicatively coupled to the communication path 104 and communicatively coupled to the one or more processors 102. Each of the one or more tactile feedback devices 107 may be any device capable of providing tactile feedback to the user. For example, one or more tactile feedback devices 107 may be embedded within the electronic device 115. In some embodiments, the one or more tactile feedback devices 107 may include a vibration device.

Referring now to FIGS. 1 and 3-5, the electronic device cover 110 is removably engageable with the electronic device 115. The gas sensor 160 may be coupled to the electronic device cover 110. Further, the control circuit 150 is communicatively coupled to the gas sensor 160 and is communicatively engageable with an electronic device 115, for example, using a wireless connection using near field communications, Bluetooth, or the like, using a wired connection, or using any of the wired or wireless connections described above. In operation, when the gas sensor 160 detects a presence of a target gas, the control circuit 150 receives a signal output by the gas sensor 160 and outputs a signal receivable by an electronic device 115. Each signal may indicate whether the target gas is present and, in some embodiments, may indicate the amount of target gas present, for example, the power level of the signal may be related to amount of target gas present, e.g., linearly, logarithmically, or the like.

The electronic device cover 110 may comprise a device facing surface 112 and an outer surface 114 opposite the device facing surface 112. The device facing surface 112 comprises the surface of the electronic device cover 110 that faces the electronic device 115 when the electronic device cover 110 is engaged with the electronic device 115 and the outer surface 114 comprises the surface of the electronic device cover 110 that faces away from the electronic device 115 when the electronic device cover 110 is engaged with the electronic device 115. In some embodiments, the gas sensor 160 may be coupled to the device facing surface 112 of the electronic device cover 110 and a gas access hole 180 extends from the outer surface 114 to the device facing surface 112 and is fluidly coupled to the gas sensor 160. Further, the electronic device cover 110 may comprise one or more plastic materials, for example, one or more chemically inert plastic materials, such as polytetrafluoroethylene (PTFE), polyimide, polycarbonate substrate, polyethylene terephthalate (PET) substrate, fluorinated ethylene propylene (FEP), polyether ether ketone (PEEK), acrylic, polypropylene (PP), or the like.

In some embodiments, the electronic device cover 110 may comprise a single cover portion and in other embodiments, the electronic device cover 110 may comprise multiple cover portions. For example, FIGS. 1 and 3-5 depict the electronic device cover 110 comprising three cover portions: a sensor housing portion 120, a sensor cover portion 130, and a device engaging portion 140; however, it should be understood that any number of cover portions are contemplated. Further, when the electronic device cover 110 comprises multiple cover portions, the cover portions may be coupled together to form a single unitary structure removably engageable with the electronic device 115. Moreover, each individual cover portion may comprise a device facing surface opposite an outer surface. Each device facing surface faces towards the electronic device 115 when the electronic device cover 110 is engaged with the electronic device 115 and each outer surface faces away from the electronic device 115 when the electronic device cover 110 is engaged with the electronic device 115.

The electronic device cover 110 may comprise one or more engagement features 126 (FIG. 5) sized and configured to engage the electronic device 115. For example, the one or more engagement features 126 may comprise an edge feature extending around a perimeter of the electronic device cover 110, for example, a lip portion, or the like engageable with the electronic device 115 to couple the electronic device cover 110 to the electronic device 115. It should be understood that the depicted engagement features 126 comprise example engagement features 126 and in other embodiments the one or more engagement features 126 may comprise any feature engageable the electronic device 115, for example, latches, magnets, snap-fit engagements, or the like. Moreover, it should be understood that in embodiments comprising multiple cover portions, each cover portion may comprise one or more engagement features 126.

Referring still to FIGS. 1 and 3-5, the sensor housing portion 120 of the electronic device cover 110 may be engageable with the sensor cover portion 130. Further, the sensor housing portion 120 may provide a housing location and/or a mounting location for the gas sensor 160 and the sensor cover portion 130 is engageable with the sensor housing portion 120 to cover the gas sensor 160. For example, when the electronic device cover 110 is engaged with the electronic device 115, the sensor housing portion 120 may be positioned between the electronic device 115 and the sensor cover portion 130.

The sensor housing portion 120 of the electronic device cover 110 comprises a device facing surface 122 opposite an outer facing surface 124. The sensor housing portion 120 may also comprise a sensor access feature 125 which may comprise a sensor access hole extending through the sensor housing portion 120, a sensor receiving recess extending into the device facing surface 112 or the outer facing surface 124 of the electronic device cover 110, and/or any feature structurally configured to provide gas access to one or more gas sensors 160 coupled to the sensor housing portion 120. For example, when the sensor access feature 125 comprises a sensor access hole, the gas sensor 160 may be coupled to the device facing surface 122 of the sensor housing portion 120 such that at least a portion of the gas sensor 160 is aligned with the sensor access hole. Further, when the sensor access feature 125 comprises a sensor receiving recess extending into the device facing surface 112 or the outer facing surface 124 of the electronic device cover 110, the gas sensor 160 may be positioned within sensor access feature 125 comprising a sensor receiving recess, for example, as depicted in FIG. 5.

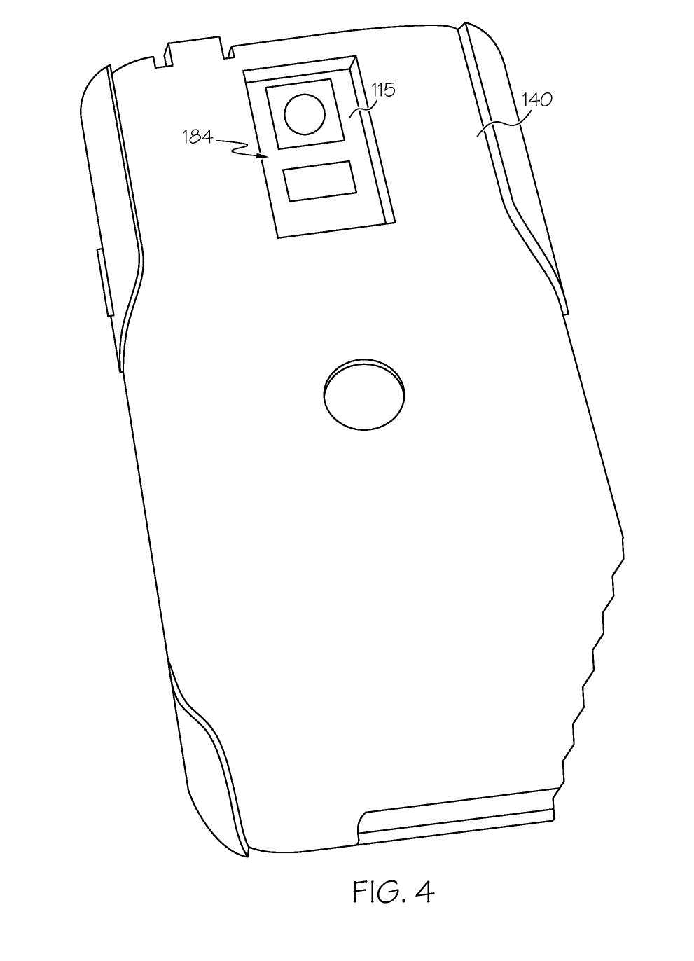

In some embodiments, the sensor housing portion 120 may directly engage with the electronic device 115. In other embodiments, as depicted in FIGS. 4 and 5, the electronic device cover 110 may further comprise a device engaging portion 140 engageable with the electronic device cover 110 and positioned between and engaged with the electronic device 115 and the sensor housing portion 120 when the electronic device cover 110 is engaged with the electronic device 115. Further, the device engaging portion 140 comprises a device facing surface 142 opposite an outer surface 144.

Referring now to FIGS. 1 and 5, the sensor cover portion 130 of the electronic device cover 110 comprises a device facing surface 132 opposite an outer surface 134. The sensor cover portion 130 may further comprise one or more gas access holes 180 extending through the sensor cover portion 130 to provide a gas pathway between the outside environment and the one or more gas sensors 160. Each of the one or more gas access holes 180 may be positioned such that when the sensor cover portion 130 is engaged with the sensor housing portion 120, the gas access hole 180 is fluidly coupled to the gas sensor 160. Further, a filter assembly 182 may be fluidly coupled to the gas sensor 160, for example, positioned within the gas access hole 180 of the sensor cover portion 130. In some embodiments, the filter assembly 182 is compositionally and structurally configured to permit target gas passage through the filter assembly and inhibit at least one other gas from traversing the filter assembly 182 and in some embodiments, the filter assembly 182 may compositionally and structurally configured to absorb heat, water vapor, or a combination thereof. For example, the filter assembly 182 may comprise any of the filter assemblies described in the patent documents incorporated by reference below, for example, Nafion™, porous polytetrafluoroethylene (PTFE), carbon, impregnated carbon cloth, KMnO₄, KMnO₄ on alumina, C/KMnO₄, triethanolamine on a silica support, or combinations thereof.

Referring now to FIG. 3, a battery 154 may be coupled to the electronic device cover 110, for example the sensor housing portion 120. Further, the battery 154 may be electrically coupled to the control circuit 150 and, in some embodiments, may be electrically coupled to the gas sensor 160 and may provide power to one or more of the components of the electronic device cover system 100. The battery 154 may comprise any battery structurally configured to power the control circuit 150 and in some embodiments, power the gas sensor 160. In some embodiments, the battery 154 may comprise one or more batteries of the electronic device 115. Further, in some embodiments, the components of the electronic device cover system 100, for example, the control circuit 150, the gas sensor 160, or the like, may be powered using a near field communication charging system, a solar charging system (e.g., one or more solar panels may be communicatively coupled to the control circuit 150 and the gas sensor 160), or any other charging systems and mechanisms. However, it should be understood that some embodiments of the gas sensor 160 may not require an external power source.

Further, as depicted in FIG. 5, the electronic device cover system 100 may comprise one or more electromagnetic shielding plates 156 coupled to the electronic device cover 110, for example, coupled the device facing surface 132 of the sensor cover portion 130 and configured to shield the control circuit 150 and the gas sensor 160 from radio waves and other electromagnetic radiation. Moreover, as depicted in FIGS. 1, 3, and 4, the electronic device cover 110 may comprise one or more device component bores 184 extending through the one or more cover portions and the electronic device cover 110. The device component bores 184 are configured to provide one or more through holes located proximate one or more ports, buttons, camera lenses, or other components of the electronic device 115 such that these components of the electronic device cover 110 are accessible to a user when the electronic device cover 110 is coupled to the electronic device cover 110.

Referring again to FIG. 5, the electronic device cover 110 may include one or more device coupling plugs 190 electrically and/or communicatively coupled to the control circuit 150 and the one or more gas sensors 160. When the electronic device cover 110 is coupled to an electronic device 115, the device coupling plugs 190 may be electrically coupled to one or more receiving ports 192 (e.g., plugs, pins, or the like) of the electronic device 115, electrically coupling the electronic device cover 110 and the electronic device 115. For example, the one or more device coupling plugs 190 extend into the electronic device 115 when the electronic device cover 110 is engaged with the electronic device 115 to form a communication pathway between the one or more gas sensors 160 and the electronic device 115. Further, while the device coupling plugs 190 extend from the device engaging portion 140 in FIG. 5, it should be understood that the device coupling plugs 190 may be positioned at any location of the electronic device cover 110 such that the device coupling plugs 190 may engage with the one or more receiving ports 192 of the electronic device 115.

Moreover, while the present disclosure refers to the electronic device cover 110 including the one or more gas sensors 160, in alternative embodiments, the electronic device cover 110 including the one or more gas sensors 160 may be directly integrated into electronic device 115, for example, as a housing of the electronic device 115. In this alternative embodiment, the gas sensors 160 may be housed with the electronic device 115 and the control circuit 150 may be one or more circuits of the electronic device 115. Further, in this embodiment, the one or more gas access holes 180 may extend into the housing of the electronic device 115 to provide gas access to the one or more gas sensors 160.

Referring again to FIG. 3, the one or more gas sensors 160 may comprise a printed gas sensor, a microelectromechanical (MEM) gas sensor, an electrochemical gas sensor, a heated metal oxide sensor, an infrared sensor, or the like, for example the printed gas sensors disclosed in U.S. patent application Ser. No. 14/317,222 titled “Printed Gas Sensor,” which is incorporated herein by reference, the printed gas sensors disclosed in U.S. Provisional Patent Application No. 62/028,543 titled “Printed Gas Sensor,” hereby incorporated by reference, and U.S. patent application Ser. No. 13/868,583 titled “Apparatus and Method for Microfabricated Multi-Dimensional Sensors and Sensing Systems,” hereby incorporated by reference. For example, the one or more gas sensors 160 may comprise gas sensors that measure a presence of the target gas in the gas sample and in some embodiments the gas sensor 160 measure an amount and/or concentration of target gas in the gas sample. As an example and not a limitation, the target gas may comprise alcohol, ethanol and/or other hydrocarbons, Ketone, CO, OH—, CH₃, CH₄, CO₂, O₃, H₂, NO, NO₂, SO₂, CH₄, O₂, H₂S, other electrochemical compounds, and combinations thereof. Further, the one or more gas sensors 160 may comprise a MEMs sensor, an SHO₂ sensor for hydrocarbons, combustibles, or the like. Further, the gas sensor 160 may comprise a volume of about 250 mm³ or less, for example, 200 mm³, 150 mm³, 100 mm³, 50 mm³, or the like.

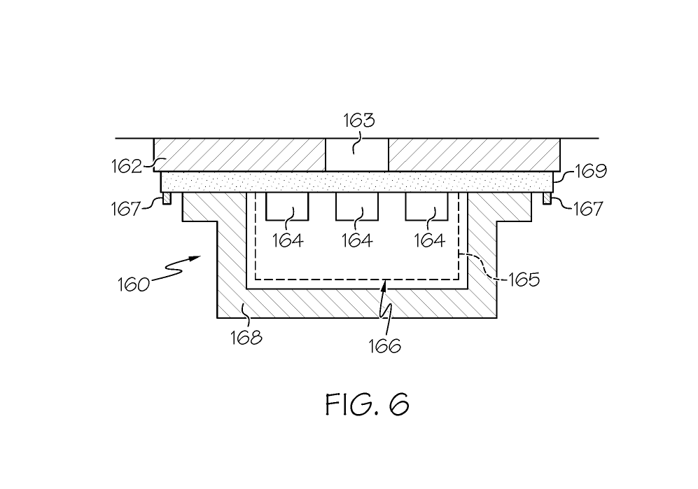

Referring now to FIG. 6, as an example and not a limitation, some embodiments of the gas sensor 160 are described below, although any exemplary sensor is contemplated. As depicted in FIG. 6, the gas sensor 160 may comprise a substrate layer 162 (e.g., a porous substrate or a partially porous substrate), one or more electrodes 164, an electrolyte cavity 166 or layer that houses liquid or gel electrolyte in electrolytic contact with the one or more electrodes 164, and an encapsulation layer 168. In some embodiments, the substrate layer 162 may be fluidly coupled to the gas access holes 180 of the electronic device cover 110 to allow the target gas to traverse the electronics device cover 110 and enter the one or more gas sensors 160 and can be any shape and size.

The substrate layer 162 may comprise one or more partially porous substrates coupled together using pressure sensitive adhesive, or the like. The substrate layer 162 may comprise low temperature plastics such as polycarbonate substrate and PET, and/or high temperature material such as PTFE, porous PTFE, or polyimide. The encapsulation layer 168 may comprise a tetrafluoroethylene (TFE) substrate, or other plastic and can be utilized to block gas access. In some embodiments, the filter assembly 182 is positioned on the substrate layer 162 such that the gas sample must pass through the filter assembly 182 before traversing the one or more gas access regions of the substrate layer 162.

The one or more electrodes 164 may be coupled to a wick 165 comprising porous glass fiber or glass fiber filter paper or may be coupled directly to the substrate layer 162. The one or more electrodes 164 may be screen printed, inkjet printed, stamped, or stenciled onto the wick 165 or substrate layer 162. The substrate layer 162 may further comprise a printed runner 169 facing the electrolyte cavity 166. The electrolyte cavity 166 may house an electrolyte, for example H₂SO₄. The one or more electrodes 164 may comprise PTFE liquid, PTFE powder, polypropylene powder, and/or polyethylene powder, as well as catalyst, solvents, and additives, such as, for example, platinum, palladium, or alloys or supported catalysts like platinum on carbon. In some embodiments, multiple electrodes 164 may be configured to each detect different target gases. For example, a first electrode can detect CO and a second electrode can detect gases such as H₂S, O₃, SO₄, or NO₂. In some embodiments, the one or more electrodes 164 are curable at temperatures lower than the melting point and deformation point of the materials of the gas sensor 160.

In operation, the electrochemical reaction between the electrode 164, the electrolyte, and the target gas generates an electric current in the printed runner 169 and sends electric signal to one or more circuits, e.g., the control circuit 150, connected to the printed runner 169 at one or more electrical contact points 167. The one or more electrical contact points 167 may be communicatively coupled to the communication path 104 such that the signal may be transmitted to the control circuit 150 and transmitted to the electronic device 115. In some embodiments, the gas sensors 160 may be electrically and communicatively coupled to the control circuit 150 using any exemplary coupling method, for example, using vias, plugs, pins, solderballs, or the like.

The electric signal output by the gas sensor 160 communicates to the control circuit 150 that a target gas is detected in the gas sensor 160 and may communicate other information regarding the target gas, for example, concentration, or the like. Further, the gas sensor 160 may output a signal at about 100 μW or less when a target gas is present within the gas sensor 160, for example, 90 μW, 75 μW, 50 μW, 25 μW, or the like. In some embodiments, the one or more gas sensors 160 may comprise a first gas sensor structurally and compositionally configured to output a signal upon exposure to a first target gas and a second gas sensor structurally and compositionally configured to output a signal upon exposure to a second target gas. Moreover, it should be understood that additional gas sensors 160 are contemplated and each additional gas sensor 160 may detect the presence of a different target gas as each of the other gas sensors 160.

In some embodiments, the electronic device cover system 100 may further include a breath sampling device integrated into and/or coupled to the electronic device cover system 100, such as the breath sampling device disclosed in U.S. patent application Ser. No. 14/851,417 titled “Breath Sampling Devices and Methods of Breath Sampling Using Sensors,” hereby incorporated by reference. The breath sampling device allow the gas sensors 160 of the electronic device cover to detect and analyze a user's breath as well as detect and analyze environmental gases. For example, the breath sampling device may be coupled to the one or more gas access holes 180 of the sensor cover portion 130 to fluidly couple the breath sampling device and the one or more gas sensors 160 such that the one or more gas sensors 160 may detect and measure alcohol present on a user's breath.

In some embodiments, the electronic device cover system 100, for example, the electronic device 115, may comprise one or more mobile applications that comprise machine readable instructions stored in the one or more memory modules 103 that are executable by the one or more processors 102 such that when the one or more processors 102 receive the sensor information output by the gas sensor 160, the one or more processors 102 perform one or more functions, for example, displaying actionable information on the display 105 of the electronic device 115. In some embodiments, when executed by the one or more processors 102, the machine readable instructions cause the one or more processors 102 to receive sensor information from the gas sensor 160, generate feedback regarding a presence and/or the concentration of a target gas based on sensor information received from the gas sensor 160, and output feedback regarding the presence and/or the concentration of the target gas using the electronic device 115.

The mobile application may generate feedback regarding target gases such as CO, alcohol, any target gas listed above, or the like. Further, the feedback may be visually output using the display 105, audibly output using the auditory device 109, and/or tactilely using the tactile feedback device 107. Moreover, each of these types of feedback may be output by the electronic device 115. Further, in some embodiments, the machine readable instructions of the one or more mobile applications may further cause the electronic device 115 to generate a calibration value when the sensor information received from the gas sensor 160 indicates that target gas is not present and/or based on a user input requesting generation of the calibration value. Additional mobile application operations include temperature and/or relative humidity compensation of the received gas sensor signals.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. An electronic device cover system comprising:
an electronic device cover engageable with an electronic device;
a gas sensor coupled to the electronic device cover; and
a control circuit communicatively coupled to the gas sensor and communicatively engageable with an electronic device, wherein when the gas sensor detects a presence of a target gas, the control circuit receives a signal output by the gas sensor and outputs a signal receivable by an electronic device.

2. The electronic device cover system of claim 1, wherein:
the electronic device cover comprises a device facing surface and an outer surface opposite the device facing surface;
the gas sensor is coupled to the device facing surface of the electronic device cover; and
a gas access hole extends from the outer surface to the device facing surface and is fluidly coupled to the gas sensor.

3. The electronic device cover system of claim 1, wherein the electronic device cover comprises a sensor housing portion engageable with a sensor cover portion, wherein the gas sensor is coupled to the sensor housing portion and a gas access hole extends through the sensor cover portion such that when the sensor cover portion is engaged with the sensor housing portion, the gas access hole is fluidly coupled to the gas sensor.

4. The electronic device cover system of claim 3, wherein the gas sensor is positioned within a sensor receiving recess of the sensor housing portion.

5. The electronic device cover system of claim 3, wherein the sensor housing portion comprises a sensor access hole and the gas sensor is coupled to a device facing side of the sensor housing portion such that at least a portion of the gas sensor is aligned with the sensor access hole.

6. The electronic device cover system of claim 1, further comprising a battery coupled to the electronic device cover and electrically coupled to the control circuit.

7. The electronic device cover system of claim 1, further comprising an electromagnetic shielding plate coupled to the electronic device cover.

8. The electronic device cover system of claim 1, further comprising a filter assembly fluidly coupled to the gas sensor.

9. The electronic device cover system of claim 8, wherein the filter assembly is compositionally and structurally configured to permit target gas passage through the filter assembly and inhibit at least one other gas from traversing the filter assembly.

10. The electronic device cover system of claim 8, wherein the filter assembly is compositionally and structurally configured to absorb heat, water vapor, or a combination thereof.

11. The electronic device cover system of claim 1, wherein the gas sensor outputs a signal at about 100 μW or less when a target gas is present within the gas sensor.

12. The electronic device cover system of claim 1, wherein the gas sensor comprises a volume of about 250 mm³ or less.

13. The electronic device cover system of claim 1, wherein the target gas comprises alcohol, ethanol, Ketone, CO, OH⁻, CH₃, CH₄, CO₂, O₃, H₂, NO, NO₂, SO₂, CH₄, O₂, H₂S, or a combination thereof.

14. The electronic device cover system of claim 1, wherein the gas sensor comprises a first gas sensor structurally and compositionally configured to output a signal upon exposure to a first target gas and a second gas sensor structurally and compositionally configured to output a signal upon exposure to a second target gas.

15. The electronic device cover system of claim 1, wherein the gas sensor comprises a micromechanical gas sensor.

16. The electronic device cover system of claim 1, wherein the gas sensor comprises a printed gas sensor.

17. The electronic device cover system of claim 16, wherein the printed gas sensor comprises:
a substrate layer comprising one or more gas access regions;
one or more printed runners coupled to the substrate layer, wherein the one or more printed runners are electrically conductive;
an encapsulation layer coupled to the substrate layer and defining an electrolyte cavity positioned within the encapsulation layer;
one or more electrodes positioned in electrical communication with the one or more printed runners such that the one or more printed runners can transport an electronic signal produced by an electrochemical reaction at the one or more electrodes; and
an electrolyte housed within the electrolyte cavity.

18. The electronic device cover system of claim 1, wherein the control circuit is communicatively engageable with an electronic device wirelessly using near field communications, Bluetooth, or a combination thereof.

19. The electronic device cover system of claim 1, wherein the control circuit is communicatively engageable with an electronic device using a wired connection.

20. An electronic device cover system comprising an electronic device cover, a gas sensor and a control circuit, wherein:
the electronic device cover is engageable with an electronic device;
the electronic device cover comprises a sensor housing portion engageable with a sensor cover portion;
the gas sensor is coupled to the sensor housing portion of the electronic device cover;
the sensor cover portion of the electronic device cover comprises a gas access hole extending through the sensor cover portion such that when the sensor cover portion is engaged with the sensor housing portion, the gas access hole is fluidly coupled to the gas sensor;
the gas sensor is a printed gas sensor comprising:
a substrate layer comprising one or more gas access regions fluidly coupled to the gas access hole of the sensor cover portion;
one or more printed runners coupled to the substrate layer, wherein the one or more printed runners are electrically conductive;
an encapsulation layer coupled to the substrate layer and defining an electrolyte cavity positioned within the encapsulation layer;
one or more electrodes positioned in electrical communication with the one or more printed runners such that the one or more printed runners can transport an electronic signal produced by an electrochemical reaction at the one or more electrodes; and
an electrolyte housed within the electrolyte cavity; and
the control circuit is communicatively coupled to the one or more printed runners of the gas sensor and communicatively engageable with an electronic device such that when the gas sensor detects a presence of a target gas, the control circuit receives a signal output by the gas sensor and outputs a signal receivable by an electronic device.

21. The electronic device cover system of claim 20, further comprising a filter assembly positioned within the gas access hole of the sensor cover portion and fluidly coupled to the gas sensor.

22. The electronic device cover system of claim 20, wherein the control circuit is communicatively engageable with an electronic device wirelessly using near field communications, Bluetooth, or a combination thereof.

23. The electronic device cover system of claim 20, wherein the control circuit is communicatively engageable with an electronic device using a wired connection.

24. A electronic device cover system for generating sensor feedback, the electronic device cover system comprising:
an electronic device cover engageable with an electronic device;
a gas sensor coupled to the electronic device cover and communicatively coupled to one or more processors;
one or more memory modules communicatively coupled to the one or more processors; and
machine readable instructions stored in the one or more memory modules that, when executed by the one or more processors, causes the one or more processors to:
receive sensor information from the gas sensor;
generate feedback regarding a presence of a target gas based on sensor information received from the gas sensor; and
output feedback regarding the presence of the target gas using the electronic device.

25. The system of claim 24, wherein the feedback regarding the presence of the target gas is visually output using a display of the electronic device.

26. The system of claim 24, wherein the feedback regarding the presence of the target gas is audibly output using an auditory device of the electronic device.

27. The system of claim 24, wherein the feedback regarding the presence of the target gas is tactile output using a tactile feedback device of the electronic device.

28. The system of claim 24, wherein the electronic device outputs feedback regarding the presence of the target gas when a detected level of target gas is greater than a threshold level of target gas.

29. The system of claim 24, wherein the machine readable instructions stored in the one or more memory modules further cause the system to perform at least the following when executed by the one or more processors:
generate a calibration value when the sensor information received from the gas sensor indicates that target gas is not present.
